(19) United States
(12) Patent Application Publication
(10) Pub. No.: US 2011/0311628 A1
(43) Pub. Date: Dec. 22, 2011

(54) PULSATILE RELEASE COMPOSITION OF THERAPEUTIC AGENT

(75) Inventors: Ramesh Muthusamy, Maharashtra (IN); Mohan Gopalkrishna Kulkarni, Maharashtra (IN)

(73) Assignee: COUNCIL OF SCIENTIFIC AND INDUSTRIAL RESEARCH, New Delhi (IN)

(21) Appl. No.: 13/254,008

(22) PCT Filed: Mar. 8, 2010
(86) PCT No.: PCT/IB10/00465

§ 371 Date: Aug. 31, 2011

(30) Foreign Application Priority Data
Mar. 9, 2009 (IN) 457/DEL/2009

(51) Int. Cl. A61K 9/32; A61K 31/405; A61K 31/196; A61K 31/522; A61K 9/52; A61K 31/545; A61P 29/00; A61P 9/00; A61P 31/00; A61P 11/06; A61K 31/554; A61K 31/167

(52) U.S. Cl. 424/482; 514/211.07; 514/420; 514/567; 514/263.34; 514/629; 514/209; 427/2.21

(57) ABSTRACT

A pulsatile release composition which releases the content rapidly after a predetermined lag time comprising a drug containing a core, coated with a pH sensitive graft copolymer. The coating suppresses the drug release at acidic pH prevalent in the stomach and releases it either immediately or after a lag time in the intestinal region. Combinations of multiple numbers of coated and uncoated units provide a sequential pulsatile release of same or different therapeutic agents.

### FIELD OF THE INVENTION

The present invention relates to pulsatile release composition for oral administration comprising multiple numbers of units wherein at least one of the units is coated with pH sensitive graft copolymer. The units comprise a therapeutically active agent and pharmaceutically acceptable ingredients.

The present invention also relates to pharmaceutical composition which provides pulsatile release of the therapeutic agent after a lag time.

### BACKGROUND OF THE INVENTION

Sustained drug delivery systems are preferred since they avoid multiple dosing in a day and reduce drug toxicity. However, in some cases they are not desirable for instance, the drugs which undergo fast metabolic degradation. This effect is more pronounced when drug release takes place slowly. Similarly, drugs which cause side effects due to their longer stay in the absorption site should not be provided as a sustained release dosage form. Site specific and time controlled pulsatile drug delivery systems are desirable for these kinds of drugs.

Pulsatile drug delivery systems are also found to be useful for the treatment of diseases which follow circadian rhythm. For example in cases like cardiac arrest, bronchial asthma and rheumatoid arthritis the attacks are more likely in the early morning, post awakening period. Once in a day bedtime dose which releases the drug after a predetermined lag time is preferable to treat such kinds of diseases. These dosages are able to maintain the effective drug concentration in the early hours of the day and avoid continuous exposure of the drug to the body.

Most of the pulsatile release drug delivery systems are reservoir systems and comprise a barrier. Erosion, dissolution or disengagement of the barrier from the drug containing core, results in rapid release of the drug.

References may be made to patent application WO 90/09168, wherein a drug dispensing device has been disclosed. The device comprises an insoluble and impermeable capsule loaded with drug and orifice of the capsule sealed with plug material. The plug material is a hydrogel which swells on contacting aqueous medium and gets ejected from the capsule after the predetermined lag time, which leads to rapid release of the drug.

References may be made to patent application WO 91/12795, wherein similar device was disclosed. It comprises a water soluble or dispersible component as a plug material. Upon contact with aqueous medium the dissolution or dispersion of plug material after a delay time, releases a pulse of the drug. As disclosed, the capsules were made using thermoplastic polymers like polyethylene, polypropylene, polyvinyl chloride, polystyrene, poly tetrafluoroethylene or by using water soluble polymers coated with said thermoplastic polymers. Since the above polymers neither dissolve nor disintegrate in the body fluid, the delivery systems based on these polymers may not be biocompatible and their presence in the body for extended time periods would be undesirable.

References may be made to U.S. Pat. No. 4,865,849, wherein a multilayered tablet composition is disclosed, in which claimed the drug release in successive pulses. The tablet comprised three layers wherein the two outer layers were separated by a barrier layer. The drug was incorporated in the outer layers comprising a hydrophilic swellable polymer capable of generating disintegration force. The barrier layer comprised a gellable or soluble polymer and other ingredients. One of the outer layers and barrier layer were enclosed within a hydrophobic housing which comprised a water insoluble, impermeable polymer. Upon contact with aqueous medium, the layer left out of the housing released the drug instantaneously. The second pulse of drug release occurred after the dissolution or rupture of the barrier layer.

References may be made to U.S. Pat. No. 5,387,421, wherein a multi stage drug delivery device was disclosed. The device comprised an outer container incorporated with one portion of drug. The inner container was incorporated with other portion of drug, an osmotic agent and a reactive agent capable of increasing the internal pressure. The inner container was coated with water insoluble, permeable polymer and the open end was sealed with plug material. This inner container was plugged into the mouth of outer container in such a way that sealed end of the inner container was inside of the outer container. The first pulse release occurs immediately after the ejection of inner container which exposed the drug present in the outer container. Upon permeation of aqueous medium, osmotic pressure generated within the inner container pushed the plug material out causing the drug release in another pulse. Thickness of the coating provided on the inner container influenced the rate of aqueous medium penetration into the inner container which determined the time lapsed between the first and the second pulse.

References may be made to U.S. Pat. No. 5,017,381, wherein a dispensing device which comprised multiple units of active agents placed one after another is disclosed. One end of the dispenser contained an expandable unit, which pushed the active agent units linearly towards the opposite end. The units came out of the dispenser and released the content in the surrounding fluid. The lag time between the pulses depended upon the length of drug dosage units which influenced the movement within the dispenser. As described in the above disclosures, the fabrication of dosage forms involved multilayered construction. This required special and expensive equipments for the manufacture of dosage forms. Also multilayered dosages are not reliable in their performance as release behavior is unpredictable and may often result in incomplete drug release.

References may be made to Journal “M. E. Sangalli, A. Maroni, A. Foppoli, L. Tema, F. Giordano and A. Gazzaniga, Journal of Controlled Release, 68, 215-223, 2000” wherein a press coated timed release composition of diltiazem hydrochloride is described. It comprised a drug containing core tablet press coated with various viscosity grades of hydroxylpropyl cellulose as a barrier. The lag time for the drug release increased with increasing viscosity of hydroxylpropyl cellulose and coating thickness.

References may be made to Journal “Shan-Yang Lin, Kung-Hsu Lin and Mei-Jane Li, Journal of Controlled Release, 70, 321-328, 2001” wherein a dry coated composition of diclofenac sodium is described. Ethylcellulose having various particle sizes was dry coated on the drug containing core tablet. The lag time was longer, when particle size was small. This was attributed to the better compaction of the system which reduced the rate of fluid penetration. By coating the tablet with ethylcellulose of various particle sizes, tablets with various lag time could be obtained.

References may be made to Journal “M. Efentakis, S. Koligliati, and M. Vlachou, International Journal of Pharmaceutics, 311, 147-156, 2006” wherein a pulsatile release composition is described. The system comprised a drug containing core tablet which was dry coated with impermeable polymer by exposing only one side of the tablet. The uncoated side of the tablet was sealed with top layer comprising hydrophilic swellable polymer which included sodium carboxymethyl cellulose, Polyethylene oxide and sodium alginate. Dissolution/erosion of the top layer led to the release of the incorporated drug. The lag time before the drug release was influenced by thickness and nature of the top layer. The lig time increased with thickness of the top layer. However, the systems having higher thickness of top layer provided sustained release after a lag time which is not expected of a pulsatile delivery system.

References may be made to U.S. Pat. No. 4,871,549, wherein Ueda et al. describe a multiparticulate pulsatile release composition comprised a sugar core coated with multiple layers in the sequence of drug layer, swellable polymer layer and water insoluble, permeable polymer layer. Permeation of fluid caused the expansion of swellable layer which resulted in bursting of the outer layer and rapid release of drug. Thickness of the outer layer influenced the lag time. The lag time increased with increasing coating thickness. Mixing of appropriate combination of the coated units and packing them together as a single unit provided a multipulse release system.

References may be made to Journal “Andrei Dashevsky and Ahmad Mohamad, International Journal of Pharmaceutics, 318, 124-131, 2006” wherein a similar system wherein the outer coating was applied in the form of aqueous dispersion instead of organic solution is described. Rate of drug release after the lag time depended upon the nature of swellable polymer. For instance, cross-linked carboxymethyl cellulose resulted in faster release. Hydroxypropyl cellulose and sodium starch glycolate at low degrees of substitution resulted in sustained release after the lag time.

It is evident from the above disclosures that, by selecting appropriate combination of particles, a series of pulses can be tailored in the case of multiparticulate systems. However a thicker coating is necessary in order to achieve desirable lag time. Failure of such coating to undergo rupture may lead to incomplete drug release in the body which is not desirable. Most importantly these types of time controlled pH independent pulsatile release compositions are not reliable, since the gastric emptying time is known to vary widely among the humans. If the dosage form is retained within the stomach for a longer period of time, the drug may be released in stomach. This may cause serious side effects, especially in case of drugs which induce the inflammation in the stomach. Similarly drugs which are susceptible to degradation at the acidic pH of the stomach should not be delivered using pH independent pulsatile release systems.

In the case of pulsatile release compositions based on pH dependent anionic polymers, the drug release does not take place until near neutral or alkaline pH is reached. Chen disclosed such a pH dependent multilayered composition in U.S. Pat. No. 5,260,069 which comprised an inner layer of drug and swellable polymer layer coated on the sugar seeds. The above seeds were further coated with a mixture of water insoluble, permeable polymer, alkaline soluble polymer and a permeability reducing agent as an outer layer. By changing the composition and coating thickness the lag time could be varied. U.S. Pat. No. 5,508,040 described a similar composition which comprised an osmotic agent in the inner layer instead of swellable polymer. Also the alkaline soluble polymer in the outer layer was eliminated. Dissolution of the osmotic agent in aqueous medium brought about the rupture of outer layer and drug release.

References may be made to U.S. Pat. No. 6,627,223 B2 A, wherein multipulse release composition comprised a sugar sphere and drug is disclosed. These spheres were coated with an enteric polymer as the first barrier membrane. The second membrane layer comprising a mixture of water insoluble polymer and an enteric polymer was applied above the first membrane. A multi pulse dosage form comprised a mixture of uncoated and membrane coated sugar spheres wherein the thickness of coating among the coated spheres was different.

References may be made to U.S. Pat. No. 6,500,457 B, wherein Midha et al. disclosed a pulsatile release composition of antiarrhythmic agents. The dosage form comprised a multiple numbers of tablets, granules or beads coated with drug and swellable polymer as an inner layer. The outer layer comprised either water soluble, water permeable or alkaline soluble polymer or combinations of them. The outer layer thickness was different for each component of the dosage. It was claimed that by choosing appropriate combination of coated units a sequence of pulse release profiles are achievable.

References may be made to Journal “T. Y. Fan, S. L. Wei, W. W. Yan, D. B. Chen and J. Li, Journal of Controlled Release, 77, 245-251, 2001” wherein A pH dependent pulsatile release composition was disclosed. The core tablet comprising a drug, swelling agent and other ingredients was coated with mixture of ethylcellulose and Eudragit L (1:2) and the coating thickness was varied. The lag time increased with coating thickness when crosslinked polyvinylpyrrolidone was used as a swelling agent in the core. However, sustained release of drug occurred after a lag time when sodium carboxymethyl starch was used as a swelling agent in the core.

References may be made to U.S. Pat. No. 6,531,152 B1, wherein a pulsatile release composition is disclosed. It comprised a drug and swelling agent in the core coated with a mixture of water insoluble, permeable polymer and hydrophilic metal salt like calcium pectinate as the first layer. The system was further coated with an enteric polymer as a second layer. The dosage form obtained could provide a single pulse release of drug after a lag time period. A little modification of the above system provides a two pulse drug delivery system as disclosed by Penhasi et al. in U.S. Pat. No. 6,632,451 B2. To the above composition an additional drug layer was added as an intermediate layer between the first and second layer. It was claimed that the dosage is intact in acidic pH condition due to the enteric coating and then gives a first pulse release immediately after placing in neutral pH condition. The first layer slowly imbibed the fluid which allowed the core to swell and burst. This led to second pulse release after a delay time period.

It is evident from the above disclosures that there is a need to design the pulsatile release compositions to treat various diseases effectively with minimized side effects. pH dependent pulsatile release compositions are desirable to target the intestinal region. Most of the currently available formulations require special and expensive equipments to manufacture them. Therefore there is a need for pH dependent pulsatile release composition which can be made relatively easily without compromising on their performance. The present invention provides such a composition.

### OBJECTIVE OF THE INVENTION

The main objective of the present invention is to provide pulsatile release composition for oral administration comprising multiple numbers of units wherein at least one of the units is coated with pH sensitive graft copolymer.

Another objective of the present invention is to provide pulsatile release composition comprising multiple numbers of units wherein each unit comprises a therapeutically active agent and pharmaceutically acceptable ingredients.

Another objective of the present invention is to provide pharmaceutical composition which provides pulsatile release of the therapeutic agent after a lag time.

### SUMMARY OF THE INVENTION

Accordingly, present invention provides a pulsatile release composition for oral administration comprising therapeutically active agent and pharmaceutically acceptable ingredients as a core unit and the said core unit is coated with pH sensitive graft copolymer having the formula 1

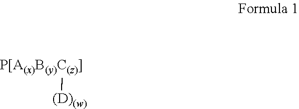

comprises:
a. a backbone having the formula P [A₍ₓ₎ B_{(y)} C_{(z)}] comprising: a diol (A), a dicarboxylic acid or acid anhydride (B) and a monomer containing pendent unsaturation (C) wherein (x)=39-45%, (y)=49-53%, (z)=5-8% by mole;
b. a graft which is a polymer of acidic monomer (D) comprises ‘w’ weight percent of the total weight of said graft copolymer such that ‘w’ is 27-56%.
   and a plasticizer.

In an embodiment of the present invention, each unit comprises a therapeutically active agent and pharmaceutically acceptable ingredients.

In yet another embodiment of the present invention, the backbone is poly (ester-ether) or polyester.

In yet another embodiment of the present invention, the diol is selected from the group consisting of aliphatic diol, cycloaliphatic diol and aromatic diol.

In yet another embodiment of the present invention, the aliphatic diol is selected from the group consisting of diethylene glycol, triethylene glycol, tetraethylene glycol, dipropylene glycol, tripropylene glycol, polyethylene glycol (Mₙ˜200), polyethylene glycol (Mₙ˜400), polyethylene glycol (Mₙ˜1000), polyethylene glycol (Mₙ˜2000), 1,2-ethane diol, 1,3-propane diol; 1,2-propane diol, 2-methyl-1,3-propane diol, 1,4-butane diol, 1,3-butane diol, 1,2-butane diol, 1,5-pentane diol, 1,6-hexane diol, 1,7-heptane diol, 1,8-octane diol, 1,9-nonane diol and 1,12-dodecane diol.

In yet another embodiment of the present invention, the cycloaliphatic diol is 1,4-cyclohexanedimethanol.

In still another embodiment of the present invention, the aromatic diol is bis(2-hydroxyethyl)terephthalate.

In yet another embodiment of the present invention, the dicarboxylic acid is selected from the group consisting of succinic acid, glutaric acid, adipic acid, pimelic acid, suberic acid, azelaic acid, sebacic acid and dodecanedioic acid.

In yet another embodiment of the present invention, the acid anhydride is selected from succinic anhydride and phthalic anhydride.

In still another embodiment of the present invention, the monomer containing pendent unsaturation is an epoxy monomer or a diol monomer.

In yet another embodiment of the present invention, the epoxy monomer is selected from glycidyl methacrylate and glycidyl acrylate.

In yet another embodiment of the present invention, the diol monomer is selected from trimethylolpropane monomethacrylate and trimethylolpropane monoacrylate.

In still another embodiment of the present invention, the acidic monomer (D) is a carboxylic acid selected from acrylic acid and methacrylic acid.

In yet another embodiment of the present invention, the plasticizer is di-n-butyl phthalate.

In yet another embodiment of the present invention, the therapeutically active agent is selected from the group consisting of, but not limited to anti-inflammatory drugs, cardiovascular drugs, antibiotic drugs, analgesic drugs and anti-asthmatic drugs.

In still another embodiment of the present invention, the anti-inflammatory drug is selected from the group consisting of but not limited to ibuprofen, ketoprofen, indomethacin, diclofenac and naproxen drugs.

In yet another embodiment of the present invention, the cardiovascular drug is selected from the group consisting of but not limited to verapamil, nifedepine, captopril, propranolol, atenolol and diltiazem.

In yet another embodiment of the present invention, the antibiotic drug is selected from the group consisting of but not limited to ampicillin and cephalexin.

In still another embodiment of the present invention, the analgesic drug is selected from the group consisting of but not limited to acetylsalicylic acid, acetaminophen, oxycodone and morphine.

In yet another embodiment of the present invention, the anti-asthmatic drug is selected from the group consisting of but not limited to aminophylline, theophylline and salbutamol.

In yet another embodiment of the present invention, multiple units contain same therapeutically active agent.

In still another embodiment of the present invention, multiple units contain different therapeutically active agents.

In yet another embodiment of the present invention, pharmaceutically acceptable ingredients are selected from, but not limited to the group consisting of, filler, binder, lubricant and glidant, preservatives, colours, flavouring agents, binders.

In yet another embodiment of the present invention, filler is selected from microcrystalline cellulose and lactose monohydrate, binder is selected from hydroxypropyl methyl cellulose and polyvinylpyrrolidone, lubricant is selected from magnesium stearate and talc and glidant is aerosil.

In still another embodiment of the present invention, the pH sensitive graft copolymer coat is 7-25% of the total weight of each coated unit.

In yet another embodiment of the present invention, the plasticizer is 5-30% of the weight of the pH sensitive graft copolymer of the invention.

In still another embodiment of the present invention, the therapeutically active agent comprises 20-70% of the total weight of each unit.

In still another embodiment of the present invention, the pharmaceutically acceptable ingredients comprise 20-60% of the total weight of each unit.

In yet another embodiment of the present invention, the pharmaceutically acceptable ingredients comprise filler 9-54%, a binder 5-15%, a lubricant 0.5-2% and a glidant 0.2-1% of the total weight of each unit.

In yet another embodiment of the present invention, the units are tablets.

In yet another embodiment of the present invention, process for the preparation of pulsatile release composition comprises the steps of:
I. dry granulating and mixing the therapeutically active agent and pharmaceutically acceptable ingredients to obtain granular mixture;
II. compressing the granular mixture as obtained in step (I) into tablets;
III. dissolving pH sensitive graft copolymer and plasticizer in solvent mixture to obtain 10% solution;
IV. coating the tablets as obtained in step (II) with 10% solution of the pH sensitive graft copolymer and plasticizer as obtained in step (III) to obtain coated tablets;
V. drying the coated tablets as obtained in step (IV) to obtain pulsatile release composition for oral administration.

In yet another embodiment of the present invention, pulsatile release composition which is capable of releasing the drug in a pulsed manner either immediately or after a lag time is provided.

In yet another embodiment of the present invention, a pulsatile release composition of one or more than one therapeutic agents is provided.

In still another embodiment of the present invention, a pulsatile release composition which is capable of protecting the drug from the acidic environmental condition is provided.

In still another aspect of the invention, a pulsatile release composition which is capable of avoiding the gastric inflammation is provided.

### BRIEF DESCRIPTION OF THE DRAWINGS

FIG. 1 shows sequential pulse release profile of the formulation comprising indomethacin.

FIG. 2 shows sequential pulse release profile of the formulation comprising acetaminophen and diclofenac sodium.

### DETAILED DESCRIPTION OF THE INVENTION

The present invention provides a pulsatile release composition which comprises;

(a) a multiple numbers of units wherein at least one of the units is coated with pH sensitive graft copolymer having the formula 1 which comprises;

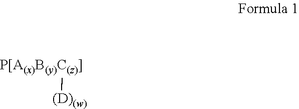

(i) a backbone having the formula P [A₍ₓ₎ B_{(y)} C_{(z)}] comprising (A) a diol, (B) a dicarboxylic acid or acid anhydride and (C) a monomer containing pendent unsaturation wherein (x)=39-45%, (y)=49-53%, (z)=5-8% by mole; and
(ii) a graft which is a polymer of acidic monomer (D) which comprises ‘w’ weight percent of the total weight of said graft copolymer such that ‘w’ is 27-56%
(b) a therapeutically active agent; and
(c) optionally pharmaceutically acceptable ingredients.

The backbone is poly (ester-ether) or polyester.

The diol (A) is selected from the group comprising aliphatic diol, cycloaliphatic diol and aromatic diol. The aliphatic diol is selected from diethylene glycol, triethylene glycol, tetraethylene glycol, dipropylene glycol, tripropylene glycol, polyethylene glycol (Mₙ˜200), polyethylene glycol (Mₙ˜400), polyethylene glycol (Mₙ˜1000), polyethylene glycol (Mₙ˜2000), 1,2-ethane diol, 1,3-propane diol, 1,2-propane diol, 2-methyl-1,3-propane diol, 1,4-butane diol, 1,3-butane diol, 1,2-butane diol, 1,5-pentane diol, 1,6-hexane diol, 1,7-heptane diol, 1,8-octane diol, 1,9-nonane diol and 1,12-dodecane diol. The cycloaliphatic diol is 1,4-cyclohexanedimethanol. The aromatic diol is bis(2-hydroxyethyl) terephthalate.

The dicarboxylic acid or acid anhydride (B) is selected from succinic acid, glutaric acid, adipic acid, pimelic acid, suberic acid, azelaic acid, sebacic acid, dodecanedioic acid, succinic anhydride and phthalic anhydride.

The monomer containing pendent unsaturation (C) is selected from glycidyl methacrylate, glycidyl acrylate, trimethylolpropane monomethacrylate and trimethylolpropane monoacrylate.

The acidic monomer (D) is selected from acrylic acid and methacrylic acid.

The pulsatile release composition comprises at least one therapeutic agent which is selected from, but not limited to the group consisting of anti-inflammatory drugs such as ibuprofen, ketoprofen, indomethacin, diclofenac and naproxen, cardiovascular drugs such as verapamil, nifedepine, captopril, propranolol, atenolol and diltiazem, antibiotic drugs such as ampicillin and cephalexin, analgesic drugs such as acetylsalicylic acid, acetaminophen, oxycodone and morphine, anti-asthmatic drugs such as aminophylline, theophylline and salbutamol.

The pulsatile release composition further comprises pharmaceutically acceptable ingredients selected form, but not limited to the group comprising filler, binder, lubricant and glidant.

The oral dosage form of pulsatile release composition is tablets. The tablet comprises therapeutically active agent in the range of 20-70% and the pharmaceutically acceptable ingredients in the range of 20-60% of the total weight of tablet. The pharmaceutically acceptable ingredients comprise; a filler 9-54% (e.g., microcrystalline cellulose, lactose monohydrate), a binder 5-15% (e.g., Hydroxypropyl methyl cellulose 5 Cps, polyvinylpyrrolidone K 30), a lubricant 0.5-2% (e.g., Magnesium stearate, talc) and a glidant 0.2-1% (e.g., aerosil) of the total weight of tablet.

The development of pulsatile release composition involves preparation of tablets using the above composition and coating them with said pH sensitive graft copolymer. The tablet is prepared by suitable granulating methods such as dry granulation of drug. To this a lubricant and glidant were added and mixed thoroughly. The granular mixture was compressed into tablets of desired size such as 8 mm diameter tablet. The tablet was coated with the said pH sensitive graft copolymer (7-25% of the total weight of each coated tablet). The coating composition also comprised a plasticizer 15% (e.g., Di-n-butyl phthalate) on the weight of said coat.

Each of the coated tablets is capable of providing a pulse release after a lag time. Combination of multiple numbers of coated tablets and optionally uncoated tablets provide a composition which is capable of providing sequential pulse releases. One such combination comprises multiple numbers of tablets wherein the therapeutic agent is same while the composition of coated pH sensitive graft copolymers is different among the tablets. Another such combination comprises multiple numbers of tablets wherein the therapeutic agents are different while the composition of coated pH sensitive graft copolymer is same among the tablets. This composition provides sequential pulse release of more than one drug.

In the examples to describe diol, dibasic acid, unsaturated monomer and acidic monomer the following abbreviations are used.

1,4 BD—1,4 Butane diol, 1,6 HD—1,6 Hexane diol, EG—Diethylene glycol, 1,4 CD—1,4 Cyclohexane dimethanol, BHET—bis(2-hydroxyethyl) terephthalate, SA—Succinic acid, SEB—Sebasic acid, AA—Adipic acid, DDA—Dodecanedioic acid, PA—Phthalic anhydride, GMA—Glycidyl methacrylate, TMPMA—Trimethylolpropane monomethacrylate, MAA—Methacrylic acid.

The synthesis of pH sensitive graft copolymers based on the above monomers is described in our co pending application 0452DEL2009 which is included herein fully as reference.

The following examples are given by way of illustration only and therefore should not be construed to limit the scope of the present invention.

### Example 1

This example describes the preparation and the dissolution profile of diltiazem hydrochloride tablet comprising the pH sensitive graft copolymer

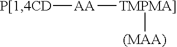

in the coat wherein the MAA content is 35 wt. %.

### (a) Preparation of Tablet

The tablet was prepared by dry granulation method. Diltiazem hydrochloride, lactose monohydrate and hydroxypropyl methyl cellulose (5 Cps) were dry granulated. To this magnesium stearate and aerosil were added and mixed thoroughly. The composition was compressed into the tablet. The diameter of the tablet was 8.0 mm. The composition of tablet is given in Table 1.

TABLE 1
The composition of tablet

| Tablet composition | Weight (mg) |
|---|---|
| Diltiazem hydrochloride | 60.00 |
| Lactose monohydrate | 108.00 |
| Hydroxypropyl methyl cellulose (5 Cps) | 10.00 |
| Magnesium stearate | 1.00 |
| Aerosil | 1.00 |
| Total | 180.00 |

### (b) Preparation of Coated Tablet

The tablet was coated with the pH sensitive graft copolymer. 10% polymer solution was prepared by dissolving the polymer and di-n-butyl phthalate in chloroform/methanol solvent mixture (7:3 v/v). The solution was coated on the said tablet and dried at room temperature. The composition of coated tablet is given in Table 2.

TABLE 2
The composition of coated tablet

| Coated tablet composition | Weight (mg) |
|---|---|
| Tablet | 180.00 |
| Graft copolymer | 20.00 |
| Di-n-butyl phthalate | 3.00 |
| Total | 203.00 |

(c) Dissolution of Diltiazem Hydrochloride from the Coated Tablet

Dissolution of diltiazem hydrochloride from the coated tablet was carried out in USP dissolution apparatus using paddle method. The paddle rotation speed was 50 rpm and the temperature was maintained at 37±0.5° C. The tablet was exposed to dissolution medium of 0.1 N HCl for the first two hours and then pH 6.8 phosphate buffer solution. The cumulative percent release of diltiazem hydrochloride is given in Table 3.

TABLE 3
Dissolution of diltiazem hydrochloride from the coated tablet

| Time (hrs.) | 1.0 | 2.0 | 2.5 | 3.0 | 3.5 | 4.0 | 4.5 | 5.0 | 5.5 | 6.0 |
|---|---|---|---|---|---|---|---|---|---|---|
| % Dissolved | 1.22 | 1.23 | 2.44 | 2.45 | 2.75 | 4.82 | 50.51 | 95.55 | 98.06 | 98.52 |
| (cumulative) | | | | | | | | | | |

### Example 2

This example describes the preparation and the dissolution profile of diltiazem hydrochloride tablet comprising the pH sensitive graft copolymer

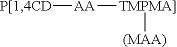

### (a) Preparation of Tablet

The tablet was prepared by dry granulation method. Diltiazem hydrochloride, lactose monohydrate and hydroxypropyl methyl cellulose (5 Cps) were dry granulated. To this magnesium stearate and aerosil were added and mixed thoroughly. The composition was compressed into the tablet. The diameter of the tablet was 8.0 mm. The composition of tablet is given in Table 4.

TABLE 4
The composition of tablet

| Tablet composition | Weight (mg) |
|---|---|
| Diltiazem hydrochloride | 60.00 |
| Lactose monohydrate | 108.00 |
| Hydroxypropyl methyl cellulose (5 Cps) | 10.00 |
| Magnesium stearate | 1.00 |
| Aerosil | 1.00 |
| Total | 180.00 |

### (b) Preparation of Coated Tablet

The tablet was coated with the pH sensitive graft copolymer. 10% polymer solution was prepared by dissolving the polymer and di-n-butyl phthalate in chloroform/methanol solvent mixture (7:3 v/v). The solution was coated on the said tablet and dried at room temperature. The composition of coated tablet is given in Table 5.

TABLE 5
The composition of coated tablet

| Coated tablet composition | Weight (mg) |
|---|---|
| Tablet | 180.00 |
| Graft copolymer | 20.00 |
| Di-n-butyl phthalate | 3.00 |
| Total | 203.00 |

(c) Dissolution of Diltiazem Hydrochloride from the Coated Tablet

Dissolution of diltiazem hydrochloride from the coated tablet was carried out in USP dissolution apparatus using paddle method. The paddle rotation speed was 50 rpm and the temperature was maintained at 37±0.5° C. The tablet was exposed to dissolution medium of 0.1 N HCl for the first two hours and then pH 6.8 phosphate buffer solution. The cumulative percent release of diltiazem hydrochloride is given in Table 6.

TABLE 6
Dissolution of diltiazem hydrochloride from the coated tablet

| Time (hrs.) | 1.0 | 2.0 | 2.5 | 3.0 | 3.5 | 4.0 | 4.5 | 5.0 | 5.5 | 6.0 |
|---|---|---|---|---|---|---|---|---|---|---|
| % Dissolved | 0.30 | 0.91 | 1.83 | 1.53 | 83.87 | 94.99 | 95.29 | 97.14 | 97.45 | 98.68 |
| (cumulative) | | | | | | | | | | |

### Example 3

This example describes the preparation and the dissolution profile of diltiazem hydrochloride tablet comprising the pH sensitive graft copolymer

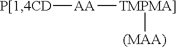

in the coat wherein the MAA content is 47 wt. %.

### (a) Preparation of Tablet

The tablet was prepared by dry granulation method. Diltiazem hydrochloride, lactose monohydrate and hydroxypropyl methyl cellulose (5 Cps) were dry granulated. To this magnesium stearate and aerosil were added and mixed thoroughly. The composition was compressed into the tablet. The diameter of the tablet was 8.0 mm. The composition of tablet is given in Table 7.

TABLE 7
The composition of tablet

| Tablet composition | Weight (mg) |
|---|---|
| Diltiazem hydrochloride | 60.00 |
| Lactose monohydrate | 108.00 |
| Hydroxypropyl methyl cellulose (5 Cps) | 10.00 |
| Magnesium stearate | 1.00 |
| Aerosil | 1.00 |
| Total | 180.00 |

### Preparation of Coated Tablet

The tablet was coated with the pH sensitive graft copolymer. 10% polymer solution was prepared by dissolving the polymer and di-n-butyl phthalate in chloroform/methanol solvent mixture (7:3 v/v). The solution was coated on the said tablet and dried at room temperature. The composition of coated tablet is given in Table 8.

TABLE 8
The composition of coated tablet

| Coated tablet composition | Weight (mg) |
|---|---|
| Tablet | 180.00 |
| Graft copolymer | 20.00 |
| Di-n-butyl phthalate | 3.00 |
| Total | 203.00 |

(c) Dissolution of Diltiazem Hydrochloride from the Coated Tablet

Dissolution of diltiazem hydrochloride from the coated tablet was carried out in USP dissolution apparatus using paddle method. The paddle rotation speed was 50 rpm and the temperature was maintained at 37±0.5° C. The tablet was exposed to dissolution medium of 0.1 N HCl for the first two hours and then pH 6.8 phosphate buffer solution. The cumulative percent release of diltiazem hydrochloride is given in Table 9.

TABLE 9
Dissolution of diltiazem hydrochloride from the coated tablet

| Time (hrs.) | 1.0 | 2.0 | 2.5 | 3.0 | 3.5 | 4.0 | 4.5 |
|---|---|---|---|---|---|---|---|
| % Dissolved | 0.61 | 0.76 | 4.04 | 88.59 | 97.90 | 98.42 | 99.00 |
| (cumulative) | | | | | | | |

### Example 4

This example describes the preparation and the dissolution profile of indomethacin tablet comprising the pH sensitive graft copolymer

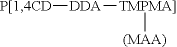

in the coat wherein the MAA content is 36 wt. %.

### (a) Preparation of Tablet

The tablet was prepared by dry granulation method. Indomethacin, lactose monohydrate and hydroxypropyl methyl cellulose (5 Cps) were dry granulated. To this magnesium stearate and aerosil were added and mixed thoroughly. The composition was compressed into the tablet. The diameter of the tablet was 8.0 mm. The composition of tablet is given in Table 10.

TABLE 10
The composition of tablet

| Tablet composition | Weight (mg) |
|---|---|
| Indomethacin | 80.00 |
| Lactose monohydrate | 47.00 |
| Hydroxypropyl methyl cellulose (5 Cps) | 20.00 |
| Magnesium stearate | 2.00 |
| Aerosil | 1.00 |
| Total | 150.00 |

### (b) Preparation of Coated Tablet

The tablet was coated with the pH sensitive graft copolymer. 10% polymer solution was prepared by dissolving the polymer and di-n-butyl phthalate in chloroform/methanol solvent mixture (7:3 v/v). The solution was coated on the said tablet and dried at room temperature. The composition of coated tablet is given in Table 11.

TABLE 11
The composition of coated tablet

| Coated tablet composition | Weight (mg) |
|---|---|
| Tablet | 150.00 |
| Graft copolymer | 50.00 |
| Di-n-butyl phthalate | 7.50 |
| Total | 207.50 |

(c) Dissolution of Indomethacin from the Coated Tablet

Dissolution of indomethacin from the coated tablet was carried out in USP dissolution apparatus using paddle method. The paddle rotation speed was 50 rpm and the temperature was maintained at 37±0.5° C. The tablet was exposed to dissolution medium of 0.1 N HCl for the first two hours and then pH 6.8 phosphate buffer solution. The cumulative percent release of indomethacin is given in Table 12.

TABLE 12
Dissolution of indomethacin from the coated tablet

| Time (hrs.) | 1.0 | 2.0 | 3.0 | 4.0 | 5.0 | 6.0 | 7.0 | 8.0 | 9.0 | 10.0 | 11.0 | 12.0 |
|---|---|---|---|---|---|---|---|---|---|---|---|---|
| % Dissolved | 0.19 | 0.19 | 0.38 | 0.58 | 0.58 | 1.74 | 2.71 | 10.87 | 34.88 | 87.46 | 97.92 | 98.82 |
| (cumulative) | | | | | | | | | | | | |

### Example 5

This example describes the preparation and the dissolution profile of indomethacin tablet comprising the pH sensitive graft copolymer

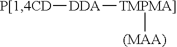

in the coat wherein the MAA content is 41 wt. %.

### (a) Preparation of Tablet

The tablet was prepared by dry granulation method. Indomethacin, lactose monohydrate and hydroxypropyl methyl cellulose (5 Cps) were dry granulated. To this magnesium stearate and aerosil were added and mixed thoroughly. The composition was compressed into the tablet. The diameter of the tablet was 8.0 mm. The composition of tablet is given in Table 13.

TABLE 13
The composition of tablet

| Tablet composition | Weight (mg) |
|---|---|
| Indomethacin | 80.00 |
| Lactose monohydrate | 47.00 |
| Hydroxypropyl methyl cellulose (5 Cps) | 20.00 |
| Magnesium stearate | 2.00 |
| Aerosil | 1.00 |
| Total | 150.00 |

### (b) Preparation of Coated Tablet

The tablet was coated with the pH sensitive graft copolymer. 10% polymer solution was prepared by dissolving the polymer and di-n-butyl phthalate in chloroform/methanol solvent mixture (7:3 v/v). The solution was coated on the said tablet and dried at room temperature. The composition of coated tablet is given in Table 14.

TABLE 14
The composition of coated tablet

| Coated tablet composition | Weight (mg) |
|---|---|
| Tablet | 150.00 |
| Graft copolymer | 50.00 |
| Di-n-butyl phthalate | 7.50 |
| Total | 207.50 |

(c) Dissolution of Indomethacin from the Coated Tablet

Dissolution of indomethacin from the coated tablet was carried out in USP dissolution apparatus using paddle method. The paddle rotation speed was 50 rpm and the temperature was maintained at 37±0.5° C. The tablet was exposed to dissolution medium of 0.1 N HCl for the first two hours and then pH 6.8 phosphate buffer solution. The cumulative percent release of indomethacin is given in Table 15.

TABLE 15
Dissolution of indomethacin from the coated tablet

| Time (hrs.) | 1.0 | 2.0 | 3.0 | 4.0 | 5.0 | 6.0 | 7.0 | 8.0 | 9.0 | 10.0 |
|---|---|---|---|---|---|---|---|---|---|---|
| % Dissolved | 0.19 | 0.38 | 0.58 | 0.77 | 1.35 | 2.71 | 9.90 | 54.69 | 92.45 | 98.37 |
| (cumulative) | | | | | | | | | | |

### Example 6

This example describes the preparation and the dissolution profile of diclofenac sodium tablet comprising the pH sensitive graft copolymer

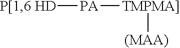

### (a) Preparation of Tablet

The tablet was prepared by dry granulation method. Diclofenac sodium, lactose monohydrate and hydroxypropyl methyl cellulose (5 Cps) were dry granulated. To this magnesium stearate and aerosil were added and mixed thoroughly. The composition was compressed into the tablet. The diameter of the tablet was 8.0 mm. The composition of tablet is given in Table 16.

TABLE 16
The composition of tablet

| Tablet composition | Weight (mg) |
|---|---|
| Diclofenac sodium | 100.00 |
| Lactose monohydrate | 37.00 |
| Hydroxypropyl methyl cellulose (5 Cps) | 20.00 |
| Magnesium stearate | 2.00 |
| Aerosil | 1.00 |
| Total | 160.00 |

### (b) Preparation of Coated Tablet

The tablet was coated with the pH sensitive graft copolymer. 10% polymer solution was prepared by dissolving the polymer and di-n-butyl phthalate in chloroform/methanol solvent mixture (7:3 v/v). The solution was coated on the said tablet and dried at room temperature. The composition of coated tablet is given in Table 17.

TABLE 17
The composition of coated tablet

| Coated tablet composition | Weight (mg) |
|---|---|
| Tablet | 160.00 |
| Graft copolymer | 40.00 |
| Di-n-butyl phthalate | 6.00 |
| Total | 206.00 |

(c) Dissolution of Diclofenac Sodium from the Coated Tablet

Dissolution of diclofenac sodium from the coated tablet was carried out in USP dissolution apparatus using paddle method. The paddle rotation speed was 50 rpm and the temperature was maintained at 37±0.5° C. The tablet was exposed to dissolution medium of 0.1 N HCl for the first two hours and then pH 6.8 phosphate buffer solution. The cumulative percent release of diclofenac sodium is given in Table 18.

TABLE 18
Dissolution of diclofenac sodium from the coated tablet

| Time (hrs.) | 1.0 | 2.0 | 3.0 | 4.0 | 5.0 | 6.0 | 7.0 | 8.0 |
|---|---|---|---|---|---|---|---|---|
| % Dissolved | 0.08 | 0.26 | 0.43 | 3.16 | 15.80 | 64.11 | 93.70 | 98.82 |
| (cumulative) | | | | | | | | |

### Example 7

This example describes the preparation and the dissolution profile of diclofenac sodium tablet comprising the pH sensitive graft copolymer

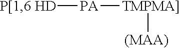

in the coat wherein the MAA content is 42 wt. %.

### (a) Preparation of Tablet

The tablet was prepared by dry granulation method. Diclofenac sodium, lactose monohydrate and hydroxypropyl methyl cellulose (5 Cps) were dry granulated. To this magnesium stearate and aerosil were added and mixed thoroughly. The composition was compressed into the tablet. The diameter of the tablet was 8.0 mm. The composition of tablet is given in Table 19.

TABLE 19
The composition of tablet

| Tablet composition | Weight (mg) |
|---|---|
| Diclofenac sodium | 100.00 |
| Lactose monohydrate | 37.00 |
| Hydroxypropyl methyl cellulose (5 Cps) | 20.00 |
| Magnesium stearate | 2.00 |
| Aerosil | 1.00 |
| Total | 160.00 |

### (b) Preparation of Coated Tablet

The tablet was coated with the pH sensitive graft copolymer. 10% polymer solution was prepared by dissolving the polymer and di-n-butyl phthalate in chloroform/methanol solvent mixture (7:3 v/v). The solution was coated on the said tablet and dried at room temperature. The composition of coated tablet is given in Table 20.

TABLE 20
The composition of coated tablet

| Coated tablet composition | Weight (mg) |
|---|---|
| Tablet | 160.00 |
| Graft copolymer | 40.00 |
| Di-n-butyl phthalate | 6.00 |
| Total | 206.00 |

(c) Dissolution of Diclofenac Sodium from the Coated Tablet

Dissolution of diclofenac sodium from the coated tablet was carried out in USP dissolution apparatus using paddle method. The paddle rotation speed was 50 rpm and the temperature was maintained at 37±0.5° C. The tablet was exposed to dissolution medium of 0.1 N HCl for the first two hours and then pH 6.8 phosphate buffer solution. The cumulative percent release of diclofenac sodium is given in Table 21.

TABLE 21
Dissolution of diclofenac sodium from the coated tablet

| Time (hrs.) | 1.0 | 2.0 | 3.0 | 4.0 | 5.0 | 6.0 | 7.0 | 8.0 | 9.0 | 10.0 |
|---|---|---|---|---|---|---|---|---|---|---|
| % Dissolved | 0.08 | 0.17 | 1.31 | 46.96 | 71.86 | 88.74 | 91.85 | 96.16 | 98.41 | 99.23 |
| (cumulative) | | | | | | | | | | |

### Example 8

This example describes the preparation and the dissolution profile of theophylline tablet comprising the pH sensitive graft copolymer

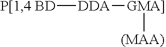

in the coat wherein the MAA content is 34 wt. %.

### (a) Preparation of Tablet

The tablet was prepared by dry granulation method. Theophylline, lactose monohydrate and hydroxypropyl methyl cellulose (5 Cps) were dry granulated. To this magnesium stearate and aerosil were added and mixed thoroughly. The composition was compressed into the tablet. The diameter of the tablet was 8.0 mm. The composition of tablet is given in Table 22.

TABLE 22
The composition of tablet

| Tablet composition | Weight (mg) |
|---|---|
| Theophylline | 120.00 |
| Lactose monohydrate | 18.00 |
| Hydroxypropyl methyl cellulose (5 Cps) | 20.00 |
| Magnesium stearate | 1.00 |
| Aerosil | 1.00 |
| Total | 160.00 |

### (b) Preparation of Coated Tablet

The tablet was coated with the pH sensitive graft copolymer. 10% polymer solution was prepared by dissolving the polymer and di-n-butyl phthalate in chloroform/methanol solvent mixture (7:3 v/v). The obtained solution was coated on the said tablet and dried at room temperature. The composition of coated tablet is given in Table 23.

TABLE 23
The composition of coated tablet

| Coated tablet composition | Weight (mg) |
|---|---|
| Tablet | 160.00 |
| Graft copolymer | 40.00 |
| Di-n-butyl phthalate | 6.00 |
| Total | 206.00 |

(c) Dissolution of Theophylline from the Coated Tablet

Dissolution of theophylline from the coated tablet was carried out in USP dissolution apparatus using paddle method. The paddle rotation speed was 50 rpm and the temperature was maintained at 37±0.5° C. The tablet was exposed to dissolution medium of 0.1 N HCl for the first two hours and then pH 6.8 phosphate buffer solution. The cumulative percent release of theophylline is given in Table 24.

TABLE 24
Dissolution of theophylline from the coated tablet

| Time (hrs.) | 1.0 | 2.0 | 3.0 | 4.0 | 5.0 | 6.0 | 7.0 | 8.0 | 9.0 |
|---|---|---|---|---|---|---|---|---|---|
| % Dissolved | 0.67 | 0.80 | 1.07 | 7.94 | 18.18 | 60.22 | 95.38 | 98.48 | 98.75 |
| (cumulative) | | | | | | | | | |

### Example 9

This example describes the preparation and the dissolution profile of theophylline tablet comprising the pH sensitive graft copolymer

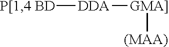

to the coat wherein the MAA content is 44 wt. %.

### (a) Preparation of Tablet

The tablet was prepared by dry granulation method. Theophylline, lactose monohydrate and hydroxypropyl methyl cellulose (5 Cps) were dry granulated. To this magnesium stearate and aerosil were added and mixed thoroughly. The composition was compressed into the tablet. The diameter of the tablet was 8.0 mm. The composition of tablet is given in Table 25.

TABLE 25
The composition of tablet

| Tablet composition | Weight (mg) |
|---|---|
| Theophylline | 120.00 |
| Lactose monohydrate | 18.00 |
| Hydroxypropyl methyl cellulose (5 Cps) | 20.00 |
| Magnesium stearate | 1.00 |
| Aerosil | 1.00 |
| Total | 160.00 |

### (b) Preparation of Coated Tablet

The tablet was coated with the pH sensitive graft copolymer. 10% polymer solution was prepared by dissolving the polymer and di-n-butyl phthalate in chloroform/methanol solvent mixture (7:3 v/v). The solution was coated on the said tablet and dried at room temperature. The composition of coated tablet is given in Table 26.

TABLE 26
The composition of coated tablet

| Coated tablet composition | Weight (mg) |
|---|---|
| Tablet | 160.00 |
| Graft copolymer | 40.00 |
| Di-n-butyl phthalate | 6.00 |
| Total | 206.00 |

(c) Dissolution of Theophylline from the Coated Tablet

Dissolution of theophylline from the coated tablet was carried out in USP dissolution apparatus using paddle method. The paddle rotation speed was 50 rpm and the temperature was maintained at 37±0.5° C. The tablet was exposed to dissolution medium of 0.1 N HCl for the first two hours and then pH 6.8 phosphate buffer solution. The cumulative percent release of theophylline is given in Table 27.

TABLE 27
Dissolution of theophylline from the coated tablet

| Time (hrs.) | 1.0 | 2.0 | 3.0 | 4.0 | 5.0 | 6.0 | 7.0 |
|---|---|---|---|---|---|---|---|
| % Dissolved | 0.80 | 0.94 | 2.02 | 28.83 | 81.64 | 97.81 | 98.62 |
| (cumulative) | | | | | | | |

### Example 10

This example describes the preparation and the dissolution profile of acetaminophen tablet comprising the pH sensitive graft copolymer

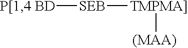

the coat wherein the MAA content is 34 wt. %.

### (a) Preparation of Tablet

The tablet was prepared by dry granulation method. Acetaminophen, lactose monohydrate and hydroxypropyl methyl cellulose (5 Cps) were dry granulated. To this magnesium stearate and aerosil were added and mixed thoroughly. The composition was compressed into the tablet. The diameter of the tablet was 8.0 mm. The composition of tablet is given in Table 28.

TABLE 28
The composition of tablet

| Tablet composition | Weight (mg) |
|---|---|
| Acetaminophen | 100.00 |
| Lactose monohydrate | 37.00 |
| Hydroxypropyl methyl cellulose (5 Cps) | 30.00 |
| Magnesium stearate | 2.00 |
| Aerosil | 1.00 |
| Total | 170.00 |

### (b) Preparation of Coated Tablet

The tablet was coated with the pH sensitive graft copolymer. 10% polymer solution was prepared by dissolving the polymer and di-n-butyl phthalate in chloroform/methanol solvent mixture (7:3 v/v). The solution was coated on the said tablet and dried at room temperature. The composition of coated tablet is given in Table 29.

TABLE 29
The composition of coated tablet

| Coated tablet composition | Weight (mg) |
|---|---|
| Tablet | 170.00 |
| Graft copolymer | 30.00 |
| Di-n-butyl phthalate | 4.50 |
| Total | 204.50 |

(c) Dissolution of Acetaminophen from the Coated Tablet

Dissolution of acetaminophen from the coated tablet was carried out in USP dissolution apparatus using paddle method. The paddle rotation speed was 50 rpm and the temperature was maintained at 37±0.5° C. The tablet was exposed to dissolution medium of 0.1 N HCl for the first two hours and then pH 6.8 phosphate buffer solution. The cumulative percent release of acetaminophen is given in Table 30.

TABLE 30
Dissolution of acetaminophen from the coated tablet

| Time (hrs.) | 1.0 | 2.0 | 3.0 | 4.0 | 5.0 | 6.0 | 7.0 |
|---|---|---|---|---|---|---|---|
| % Dissolved | 0.51 | 2.40 | 6.52 | 41.18 | 87.57 | 98.07 | 98.42 |
| (cumulative) | | | | | | | |

### Example 11

This example describes the preparation and the dissolution profile of acetaminophen tablet comprising the pH sensitive graft copolymer

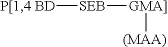

in the coat wherein the MAA content is 55 wt. %.

### (a) Preparation of Tablet

The tablet was prepared by dry granulation method. Acetaminophen, lactose monohydrate and hydroxypropyl methyl cellulose (5 Cps) were dry granulated. To this magnesium stearate and aerosil were added and mixed thoroughly. The composition was compressed into the tablet. The diameter of the tablet was 8.0 mm. The composition of tablet is given in Table 31.

TABLE 31
The composition of tablet

| Tablet composition | Weight (mg) |
|---|---|
| Acetaminophen | 100.00 |
| Lactose monohydrate | 37.00 |
| Hydroxypropyl methyl cellulose (5 Cps) | 30.00 |
| Magnesium stearate | 2.00 |
| Aerosil | 1.00 |
| Total | 170.00 |

### (b) Preparation of Coated Tablet

The tablet was coated with the pH sensitive graft copolymer. 10% polymer solution was prepared by dissolving the polymer and di-n-butyl phthalate in chloroform/methanol solvent mixture (7:3 v/v). The solution was coated on the said tablet and dried at room temperature. The composition of coated tablet is given in Table 32.

TABLE 32
The composition of coated tablet

| Coated tablet composition | Weight (mg) |
|---|---|
| Tablet | 170.00 |
| Graft copolymer | 30.00 |
| Di-n-butyl phthalate | 4.50 |
| Total | 204.50 |

(c) Dissolution of Acetaminophen from the Coated Tablet

Dissolution of acetaminophen from the coated tablet was carried out in USP dissolution apparatus using paddle method. The paddle rotation speed was 50 rpm and the temperature was maintained at 37±0.5° C. The tablet was exposed to dissolution medium of 0.1 N HCl for the first two hours and then pH 6.8 phosphate buffer solution. The cumulative percent release of acetaminophen is given in Table 33.

TABLE 33
Dissolution of acetaminophen from the coated tablet

| Time (hrs.) | 1.0 | 2.0 | 3.0 | 4.0 | 5.0 | 6.0 | 7.0 |
|---|---|---|---|---|---|---|---|
| % Dissolved | 1.37 | 1.54 | 69.89 | 96.69 | 97.90 | 98.25 | 98.59 |
| (cumulative) | | | | | | | |

### Example 12

This example describes the preparation and the dissolution profile of cephalexin monohydrate tablet comprising the pH sensitive graft copolymer

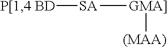

in the coat wherein the MAA content is 46 wt. %.

### (a) Preparation of Tablet

The tablet was prepared by dry granulation method. Cephalexin monohydrate, lactose monohydrate and hydroxypropyl methyl cellulose (5 Cps) were dry granulated. To this magnesium stearate and aerosil were added and mixed thoroughly. The composition was compressed into the tablet. The diameter of the tablet was 8.0 mm. The composition of tablet is given in Table 34.

TABLE 34
The composition of tablet

| Tablet composition | Weight (mg) |
|---|---|
| Cephalexin monohydrate | 140.00 |
| Lactose monohydrate | 18.00 |
| Hydroxypropyl methyl cellulose (5 Cps) | 20.00 |
| Magnesium stearate | 1.60 |
| Aerosil | 0.4 |
| Total | 180.00 |

### (b) Preparation of Coated Tablet

The tablet was coated with the pH sensitive graft copolymer. 10% polymer solution was prepared by dissolving the polymer and di-n-butyl phthalate in chloroform/methanol solvent mixture (7:3 v/v). The solution was coated on the said tablet and dried at room temperature. The composition of coated tablet is given in Table 35.

TABLE 35
The composition of coated tablet

| Coated tablet composition | Weight (mg) |
|---|---|
| Tablet | 180.00 |
| Graft copolymer | 20.00 |
| Di-n-butyl phthalate | 3.00 |
| Total | 203.00 |

(c) Dissolution of Cephalexin Monohydrate from the Coated Tablet

Dissolution of cephalexin monohydrate from the coated tablet was carried out in USP dissolution apparatus using paddle method. The paddle rotation speed was 50 rpm and the temperature was maintained at 37±0.5° C. The tablet was exposed to dissolution medium of 0.1 N HCl for the first two hours and then pH 6.8 phosphate buffer solution. The cumulative percent release of cephalexin monohydrate is given in Table 36.

TABLE 36
Dissolution of cephalexin monohydrate from the coated tablet

| Time (hrs.) | 1.0 | 2.0 | 3.0 | 4.0 | 5.0 | 6.0 |
|---|---|---|---|---|---|---|
| % Dissolved | 0.18 | 0.55 | 75.68 | 93.49 | 95.90 | 96.83 |
| (cumulative) | | | | | | |

### Example 13

This example describes the preparation and the dissolution profile of cephalexin monohydrate tablet comprising the pH sensitive graft copolymer

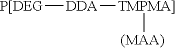

in the coat wherein the MAA content is 47 wt. %.

### (a) Preparation of Tablet

The tablet was prepared by dry granulation method. Cephalexin monohydrate, lactose monohydrate and hydroxypropyl methyl cellulose (5 Cps) were dry granulated. To this magnesium stearate and aerosil were added and mixed thoroughly. The composition was compressed into the tablet. The diameter of the tablet was 8.0 mm. The composition of tablet is given in Table 37.

TABLE 37
The composition of tablet

| Tablet composition | Weight (mg) |
|---|---|
| Cephalexin monohydrate | 140.00 |
| Lactose monohydrate | 18.00 |
| Hydroxypropyl methyl cellulose (5 Cps) | 20.00 |
| Magnesium stearate | 1.60 |
| Aerosil | 0.4 |
| Total | 180.00 |

### (b) Preparation of Coated Tablet

The tablet was coated with the pH sensitive graft copolymer. 10% polymer solution was prepared by dissolving the polymer and di-n-butyl phthalate in chloroform/methanol solvent mixture (7:3 v/v). The solution was coated on the said tablet and dried at room temperature. The composition of coated tablet is given in Table 38.

TABLE 38
The composition of coated tablet

| Coated tablet composition | Weight (mg) |
|---|---|
| Tablet | 180.00 |
| Graft copolymer | 20.00 |
| Di-n-butyl phthalate | 3.00 |
| Total | 203.00 |

(c) Dissolution of Cephalexin Monohydrate from the Coated Tablet

Dissolution of cephalexin monohydrate from the coated tablet was carried out in USP dissolution apparatus using paddle method. The paddle rotation speed was 50 rpm and the temperature was maintained at 37±0.5° C. The tablet was exposed to dissolution medium of 0.1 N HCl for the first two hours and then pH 6.8 phosphate buffer solution. The cumulative percent release of cephalexin monohydrate is given in Table 39.

TABLE 39
Dissolution of cephalexin monohydrate from the coated tablet

| Time (hrs.) | 1.0 | 2.0 | 3.0 | 4.0 | 5.0 | 6.0 | 7.0 |
|---|---|---|---|---|---|---|---|
| % Dissolved | 0.18 | 0.18 | 0.37 | 62.90 | 96.45 | 97.01 | 97.38 |
| (cumulative) | | | | | | | |

### Example 14

This example describes the preparation and the dissolution profile of diltiazem hydrochloride tablet comprising the pH sensitive graft copolymer

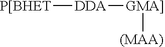

in the coat wherein the MAA content is 34 wt. %.

### (a) Preparation of Tablet

The tablet was prepared by dry granulation method. Diltiazem hydrochloride, lactose monohydrate and hydroxypropyl methyl cellulose (5 Cps) were dry granulated. To this magnesium stearate and aerosil were added and mixed thoroughly. The composition was compressed into the tablet. The diameter of the tablet was 8.0 mm. The composition of tablet is given in Table 40.

TABLE 40
The composition of tablet

| Tablet composition | Weight (mg) |
|---|---|
| Diltiazem hydrochloride | 100.00 |
| Lactose monohydrate | 37.00 |
| Hydroxypropyl methyl cellulose (5 Cps) | 30.00 |
| Magnesium stearate | 2.00 |
| Aerosil | 1.00 |
| Total | 170.00 |

### (b) Preparation of Coated Tablet

The tablet was coated with the pH sensitive graft copolymer. 10% polymer solution was prepared by dissolving the polymer and di-n-butyl phthalate in chloroform/methanol solvent mixture (7:3 v/v). The solution was coated on the said tablet and dried at room temperature. The composition of coated tablet is given in Table 41.

TABLE 41
The composition of coated tablet

| Coated tablet composition | Weight (mg) |
|---|---|
| Tablet | 170.00 |
| Graft copolymer | 30.00 |
| Di-n-butyl phthalate | 4.50 |
| Total | 204.50 |

(c) Dissolution of Diltiazem Hydrochloride from the Coated Tablet

Dissolution of diltiazem hydrochloride from the coated tablet was carried out in USP dissolution apparatus using paddle method. The paddle rotation speed was 50 rpm and the temperature was maintained at 37±0.5° C. The tablet was exposed to dissolution medium of 0.1 N HCl for the first two hours and then pH 6.8 phosphate buffer solution. The cumulative percent release of diltiazem hydrochloride is given in Table 42.

TABLE 42
Dissolution of diltiazem hydrochloride from the coated tablet

| Time (hrs.) | 1.0 | 2.0 | 3.0 | 4.0 | 5.0 | 6.0 | 7.0 | 8.0 | 9.0 |
|---|---|---|---|---|---|---|---|---|---|
| % Dissolved | 0 | 0.12 | 0.67 | 1.51 | 2.34 | 17.77 | 90.76 | 97.51 | 97.85 |
| (cumulative) | | | | | | | | | |

### Example 15

This example describes the pulsatile release composition which is capable of providing sequential pulses wherein the therapeutic agent is same and coated pH sensitive graft copolymers are different among the tablets.

Tablet 1. Coating comprises the pH sensitive graft copolymer (A)

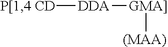

wherein the MAA content is 57 wt. %.

Tablet 2. Coating comprises the pH sensitive graft copolymer (B)

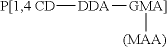

wherein the MAA content is 49 wt. %.

Tablet 3. Coating comprises the pH sensitive graft copolymer (C)

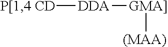

wherein the MAA content is 38 wt. %.

### (a) Preparation of Tablets

The tablets were prepared by dry granulation method. Indomethacin, lactose monohydrate and hydroxypropyl methyl cellulose (5 Cps) were dry granulated. To this magnesium stearate and aerosil were added and mixed thoroughly. The composition was compressed into the tablet. The diameter of the tablets was 8.0 mm. The compositions of tablets are given in Table 43.

TABLE 43
The composition of tablets

| | Weight (mg) | | |
|---|---|---|---|
| Tablet composition | Tablet 1 | Tablet 2 | Tablet 3 |
| Indomethacin | 100.00 | 100.00 | 100.00 |
| Lactose monohydrate | 63.00 | 47.00 | 27.00 |
| Hydroxypropyl methyl cellulose (5 Cps) | 20.00 | 20.00 | 20.00 |
| Magnesium stearate | 2.00 | 2.00 | 2.00 |
| Aerosil | 1.00 | 1.00 | 1.00 |
| Total | 186.00 | 170.00 | 150.00 |

### (b) Preparation of Coated Tablets

The tablets were coated with the pH sensitive graft copolymers A, B and C for the tablets 1, 2 and 3 respectively. 10% polymer solution was prepared by dissolving the polymer and di-n-butyl phthalate in chloroform/methanol solvent mixture (7:3 v/v). The solution was coated on the said tablets and dried at room temperature. The composition of coated tablets is given in Table 44.

TABLE 44
The composition of coated tablets

| | Weight (mg) | | |
|---|---|---|---|
| Coated tablets composition | Tablet 1 | Tablet 2 | Tablet 3 |
| Tablets | 186.00 | 170.00 | 150.00 |
| Graft copolymer (A, B and C)* | 14.00 | 30.00 | 50.00 |
| Di-n-butyl phthalate | 2.10 | 4.50 | 7.50 |
| Total | 202.10 | 204.50 | 207.50 |

*Graft copolymers A, B and C were coated on the tablets 1, 2 and 3 respectively.

(II) Dissolution of Indomethacin from Pulsatile Release Composition

The dissolution of pulsatile release composition was carried out in USP dissolution apparatus using paddle method. The paddle rotation speed was 50 rpm and the temperature was maintained at 37±0.5° C. The prepared tablet units were exposed to dissolution medium of 0.1 N HCl for the first two hours and then pH 6.8 phosphate buffer solution. The cumulative percent release of indomethacin is given in Table 45.

TABLE 45
Dissolution data of indomethacin from
the pulsatile release composition

| Time (hrs.) | 0.5 | 1.0 | 1.5 | 2.0 | 2.5 | 3.0 | 3.5 | 4.0 |
|---|---|---|---|---|---|---|---|---|
| % Dissolved | 0.10 | 0.10 | 0.20 | 0.31 | 31.57 | 33.05 | 33.78 | 34.40 |
| (cumulative) | | | | | | | | |
| Time (hrs.) | 4.5 | 5.0 | 5.5 | 6.0 | 6.5 | 7.0 | 7.5 | 8.0 |
| % Dissolved | 35.02 | 51.17 | 60.50 | 65.69 | 66.52 | 66.73 | 67.25 | 68.18 |
| (cumulative) | | | | | | | | |
| Time (hrs.) | 8.5 | 9.0 | 9.5 | 10.0 | 10.5 | 11 | 11.5 | 12.0 |
| % Dissolved | 68.80 | 81.86 | 88.14 | 95.35 | 98.59 | 99.16 | 99.54 | 99.73 |
| (cumulative) | | | | | | | | |

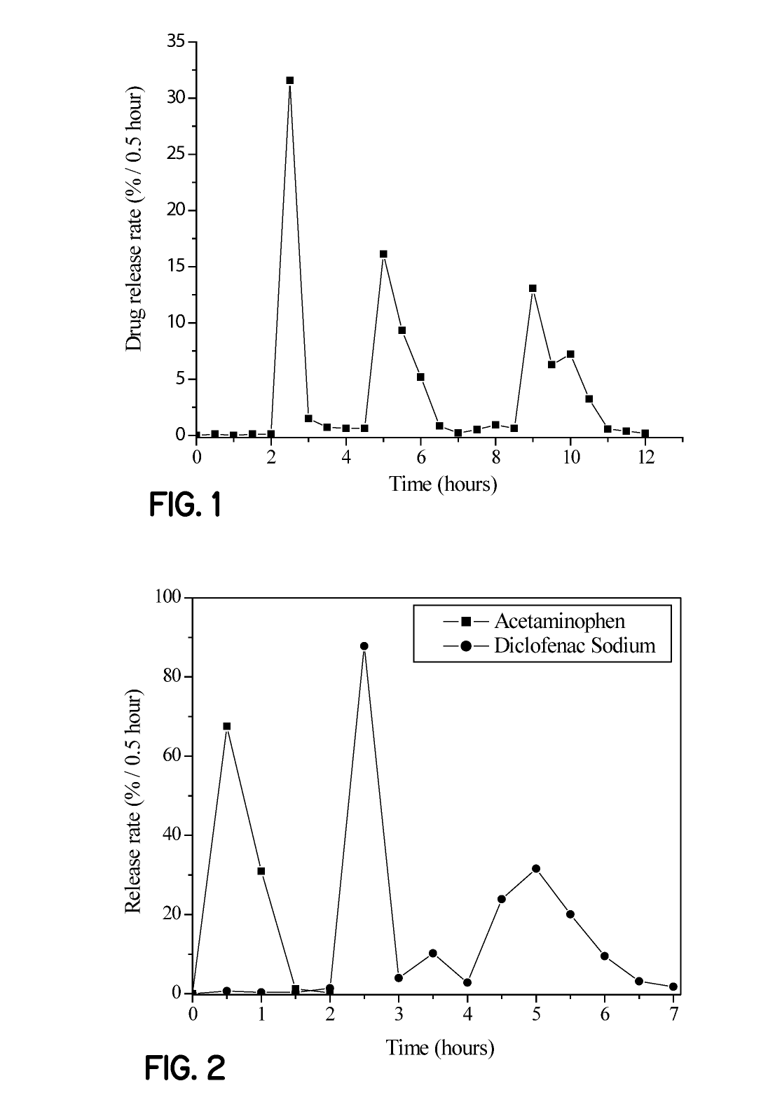

Sequential pulse release profile of the formulation comprising indomethacin is graphically represented in FIG. 1.

### Example 16

This example describes the preparation of pulsatile release composition which is capable of providing sequential pulses wherein the pH sensitive graft copolymer is same and the therapeutic agents are different among the tablet. The pH sensitive graft copolymer is,

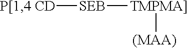

wherein the MAA content is 36 wt. %.

### 1. Preparation of Pulsatile Release Composition

### (a) Preparation of Tablets

The tablets were prepared by dry granulation method. Acetaminophen, lactose monohydrate and hydroxypropyl methyl cellulose (5 Cps) were dry granulated. To this magnesium stearate and aerosil were added and mixed thoroughly. The composition was compressed into the tablet. The diameter of the tablets was 8.0 mm. Similarly another two tablets containing diclofenac sodium were prepared. The composition of tablets is given in Table 46.

TABLE 46
The composition of tablets

| | Weight (mg) | | |
|---|---|---|---|
| Tablet composition | Tablet 1 | Tablet 2 | Tablet 3 |
| Therapeutic agent* | 150.00 | 100.00 | 100.00 |
| Lactose monohydrate | 27.00 | 63.00 | 37.00 |
| Hydroxypropyl methyl cellulose (5 Cps) | 20.00 | 20.00 | 20.00 |
| Magnesium stearate | 2.00 | 2.00 | 2.00 |
| Aerosil | 1.00 | 1.00 | 1.00 |
| Total | 200.00 | 186.00 | 160.00 |

*Tablet 1 comprises acetaminophen and tablets 2 and 3 comprise diclofenac sodium

### (b) Preparation of Diclofenac Sodium Containing Coated Tablets

The tablets were coated with the pH sensitive graft copolymer. 10% polymer solution was prepared by dissolving the polymer and di-n-butyl phthalate in chloroform/methanol solvent mixture (7:3 v/v). The solution was coated on said diclofenac sodium containing tablets and dried at room temperature. The composition of coated tablets is given in Table 47.

TABLE 47
The composition of coated tablets

| | Weight (mg) | |
|---|---|---|
| Coated tablets composition | Tablet 2 | Tablet 3 |
| Tablets | 186.00 | 160.00 |
| Graft copolymer | 14.00 | 40.00 |
| Di-n-butyl phthalate | 2.10 | 6.00 |
| Total | 202.10 | 206.00 |

### (II) Dissolution of Acetaminophen and Diclofenac Sodium Containing Pulsatile Release Composition

The dissolution of pulsatile release composition was carried out in USP dissolution apparatus using paddle method. The paddle rotation speed was 50 rpm and the temperature was maintained at 37±0.5° C. The tablet was exposed to dissolution medium of 0.1 N HCl for the first two hours and then pH 6.8 phosphate buffer solution. The cumulative percent release of acetaminophen and diclofenac sodium is given in Table 48.

TABLE 48
Dissolution data of acetaminophen and diclofenac
sodium from the pulsatile release composition

| Time (hours) | 0.5 | 1.0 | 1.5 | 2.0 | 2.0 | 3.0 | 3.5 |
|---|---|---|---|---|---|---|---|
| Dissolution of | 67.53 | 98.45 | 99.68 | 99.90 | — | — | — |
| Acetaminophen | | | | | | | |
| Dissolution of | 0.35 | 0.52 | 0.70 | 1.40 | 45.29 | 47.27 | 52.36 |
| Diclofenac sodium | | | | | | | |
| (% Cumulative) | | | | | | | |
| Time (hours) | 4.0 | 4.5 | 5.0 | 5.5 | 6.0 | 6.5 | 7.0 |
| Dissolution of | — | — | — | — | — | — | — |
| Acetaminophen | | | | | | | |
| Dissolution of | 53.77 | 65.71 | 81.52 | 91.55 | 96.30 | 97.88 | 98.76 |
| Diclofenac sodium | | | | | | | |
| (% Cumulative) | | | | | | | |

Sequential pulse release profile of the formulation comprising acetaminophen and diclofenac sodium is graphically represented in FIG. 2.

It will be evident to those skilled in the art that the invention is not limited to the details of the foregoing illustrative examples and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

### ADVANTAGES OF THE INVENTION

The disclosed formulations provide pulsatile release of the therapeutic agent after a lag time at pH prevalent in the intestinal region.
The disclosed formulations do not require more than one polymer to obtain the release as said above.
The disclosed formulations are capable of delivering more than one drug in pulsatile fashion.
This composition overcomes the disadvantages of layered dosage forms such as difficulty in formulation and swallowing.

1. A pulsatile release composition for oral administration comprising therapeutically active agent and pharmaceutically acceptable ingredients as a core unit and the said core unit is coated with pH sensitive graft copolymer having the formula 1 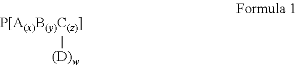
comprises:
a. a backbone having the formula P [A₍ₓ₎ B_{(y)} C_{(z)}] comprising: a diol (A), a dicarboxylic acid or acid anhydride (B) and a monomer containing pendent unsaturation (C) wherein (x)=39-45%, (y)=49-53%, (z)=5-8% by mole;
b. a graft which is a polymer of acidic monomer (D) comprises ‘w’ weight percent of the total weight of said graft copolymer such that ‘w’ is 27-56% and a plasticizer.

2. The pulsatile release composition as claimed in claim 1, wherein the backbone is poly (ester-ether) or polyester.

3. The pulsatile release composition as claimed in claim 1, wherein the diol is selected from the group consisting of aliphatic diol, cycloaliphatic diol and aromatic diol.

4. The pulsatile release composition as claimed in claim 3, wherein the aliphatic diol is selected from the group consisting of diethylene glycol, triethylene glycol, tetraethylene glycol, dipropylene glycol, tripropylene glycol, polyethylene glycol (Mₙ˜200), polyethylene glycol (Mₙ˜400), polyethylene glycol (Mₙ˜1000), polyethylene glycol (Mₙ˜2000), 1,2-ethane diol, 1,3-propane diol, 1,2-propane diol, 2-methyl-1,3-propane diol, 1,4-butane diol, 1,3-butane diol, 1,2-butane diol, 1,5-pentane diol, 1,6-hexane diol, 1,7-heptane diol, 1,8-octane diol, 1,9-nonane diol and 1,12-dodecane diol.

5. The pulsatile release composition as claimed in claim 3, wherein the cycloaliphatic diol is 1,4-cyclohexanedimethanol.

6. The pulsatile release composition as claimed in claim 3, wherein the aromatic diol is bis(2-hydroxyethyl)terephthalate.

7. The pulsatile release composition as claimed in claim 1, wherein the dicarboxylic acid is selected from the group consisting of succinic acid, glutaric acid, adipic acid, pimelic acid, suberic acid, azelaic acid, sebacic acid and dodecanedioic acid.

8. The pulsatile release composition as claimed in claim 1, wherein the acid anhydride is selected from succinic anhydride and phthalic anhydride.

9. The pulsatile release composition as claimed in claim 1, wherein the monomer containing pendent unsaturation is an epoxy monomer selected from glycidyl methacrylate and glycidyl acrylate or a diol monomer selected from trimethylolpropane monomethacrylate and trimethylolpropane monoacrylate.

10. (canceled)

11. (canceled)

12. The pulsatile release composition as claimed in claim 1, wherein the acidic monomer (D) is a carboxylic acid selected from acrylic acid and methacrylic acid.

13. The pulsatile release composition as claimed in claim 1, wherein the plasticizer is preferably di-n-butyl phthalate

14. The pulsatile release composition as claimed in claim 1, wherein the therapeutically active agent comprises anti-inflammatory drugs further comprising ibuprofen, ketoprofen, indomethacin, diclofenac and naproxen, cardiovascular drugs comprising verapamil, nifedepine, captopril, propranolol, atenolol and diltiazem, antibiotic drugs comprising ampicillin and cephalexin, analgesic drugs comprising acetylsalicylic acid, acetaminophen, oxycodone and morphine, and anti-asthmatic drugs comprising aminophylline, theophylline and salbutamol.

15. (canceled)

16. (canceled)

17. (canceled)

18. (canceled)

19. (canceled)

20. The pulsatile release composition as claimed in claim 1, wherein core unit contains same or different therapeutically active agent.

21. (canceled)

22. The pulsatile release composition as claimed in claim 1, wherein pharmaceutically acceptable ingredients are selected from, but not limited to the group consisting of, filler selected from microcrystalline cellulose and lactose monohydrate, binder selected from hydroxypropyl methyl cellulose and polyvinyl pyrrolidone, lubricant selected from magnesium stearate and talc and glidant, preservatives, colorants and flavorants.

23. The pulsatile release composition as claimed in claim 22, wherein glidant is preferably aerosil.

24. The pulsatile release composition as claimed in claim 1, wherein the pH sensitive graft copolymer coat is 7-25% of the total weight of each coated unit.

25. The pulsatile release composition as claimed in claim 1, wherein the plasticizer is 5-30% of the weight of the pH sensitive graft copolymer of claim 1 on the weight of said coat.

26. The pulsatile release composition as claimed in claim 1, wherein the therapeutically active agent comprises 20-70% of the total weight of each unit and the pharmaceutically acceptable ingredients comprises 20-60% of the total weight of each unit.

27. (canceled)

28. The pulsatile release composition as claimed in claim 1, wherein the pharmaceutically acceptable ingredients comprise filler 9-54%, a binder 5-15%, a lubricant 0.5-2% and a glidant 0.2-1% of the total weight of each unit and the units are tablets.

29. (canceled)

30. The pulsatile release composition as claimed in claim 1, wherein process for the preparation of the said composition comprises the steps of:
I. dry granulating and mixing the therapeutically active agent and pharmaceutically acceptable ingredients to obtain granular mixture;
II. compressing the granular mixture as obtained in step (I) into tablets;
III. dissolving pH sensitive graft copolymer and plasticizer in solvent mixture to obtain 10% solution;
IV. coating the tablets as obtained in step (II) with 10% solution of the pH sensitive graft copolymer and plasticizer as obtained in step (III) to obtain coated tablets;
V. drying the coated tablets as obtained in step (IV) to obtain pulsatile release composition for oral administration.
